(19) United States
(12) Patent Application Publication
(10) Pub. No.: US 2023/0092653 A1
(43) Pub. Date: Mar. 23, 2023

(54) DEVICE, SYSTEM AND METHOD FOR MEASURING MOISTURE CONTENT OF A WOOD MATERIAL

(71) Applicant: WIISTE OY, Tampere (FI)

(72) Inventors: Toni LUOPAJÄRVI, Tampere (FI); Risto-Matti SALMI, Tampere (FI)

(21) Appl. No.: 17/795,825

(22) PCT Filed: Jan. 28, 2021
(86) PCT No.: PCT/EP2021/051958

§ 371 Date: Jul. 27, 2022

(30) Foreign Application Priority Data
Jan. 28, 2020 (FI) 20205084

(51) Int. Cl. G01N 27/04; G01N 33/46

(52) U.S. Cl. CPC G01N 27/048; G01N 33/46

(57) ABSTRACT

Disclosed is a device with a data transferring unit and a method for measuring moisture content of a wood material. Knots and other imperfections in a wood material causes problems in moisture content measurements. Also, more accurate data relating to the moisture of the material is needed during the time of storing, transporting and building. A device for measuring moisture in wood material includes a body part arrangeable on a surface of the wood material, measuring unit being functionally connected to the body part, wherein the measuring unit includes at least a first pair of electrodes and a second pair of electrodes to be inserted in the wood material for investigating, and wherein a measuring depth of the first pair and the second pair is arranged to differ for providing impedance data from a first depth and/or a second depth of the wood material.

### TECHNICAL FIELD

The invention relates generally to a device, system and method for measuring moisture. More specifically the invention relates to a device with data transferring means and a method for measuring moisture content of a wood material.

### BACKGROUND TECHNOLOGY

Wood has been used as a building material for thousands of years. Wood material can be used in various kinds of forms for building houses, shelters and boats, for example, but it is also extensively used in the furniture and home decor industry as well.

The advantage of using wood material in buildings, for example, is its nature of being strong in relation to its weight, and it provides good insulation from the cold. Wood is highly machinable, and it can be fabricated into all kinds of shapes and sizes to fit practically any construction need. Wood can also be an example of an environmentally sustainable product; it is biodegradable and renewable, and it may have one of the lowest carbon foot print of any comparable building material.

However, a problem related to wood materials raises during storing and building processes. It may not always be possible to guarantee environmental circumstances dry enough for preventing the wood material from getting wet. Exposing wood to rain, snow and/or high humidity, for example, may cause moisture and/or mold damages in the wood material in question. Partly or fully wet wood material is known to facilitate abnormal microbial growth, which may be harmful for the health of human beings.

It is thus important to keep track on the moisture content of wood material during the usage in order to detect possible water damages, such as water leakage.

There are some prior known solutions for measuring moisture in wood materials. However, these inventions and methods have some limitations. such as methods disclosed in the documents GB2517172B and EP0709814A1. The documents disclose fixed electrodes for automatic monitoring of moisture in the building and in the construction material.

Unfortunately, knots in the wood cause a problem, when measuring moisture. Knots are a kind of imperfections that cause living wood grain to grow around them and the knots appear randomly in the wood. Knots in the wood are known to hinder the moisture measurement by affecting the conductivity of the wood material, especially in case where one or both electrodes are installed in a knot part.

Measuring of wood material comprising layers is also a problem. A surface layer may be exposed to moisture, while measuring device in a deeper layer does not detect any moisture, for example.

### SUMMARY OF THE INVENTION

The purpose of the present invention is to avoid or reduce the above disadvantages of the prior art. Especially the present invention tries to solve the problem how to measure the moisture content of a wood material in a reliable and feasible manner during the process of storing, transporting of wood material and constructing of a building, for example.

According to one aspect of the present invention, a device for measuring moisture in wood material is characterized in that the device comprises:
a body part arrangeable on a surface of said wood material,
measuring means being functionally connected to the body part, said measuring means comprising at least a first pair of electrodes and a second pair of electrodes to be inserted in said wood material for investigating impedance of said wood material,

wherein a measuring depth of said first pair of electrodes and said second pair of electrodes is arranged to differ for providing impedance data from a first depth and/or a second depth of said wood material.

According to another aspect of the present invention a measuring system for measuring moisture content of a wood material, is characterized in that the system comprises a measuring device according to the invention, and a remote device for receiving and collecting data related to a moisture content of said wood material.

According to a further aspect of the invention a method for moisture content of a wood material is characterized in that a moisture measuring device according to the invention is arranged on a wood material to be measured, moisture content of said wood material is measured with said moisture measuring device, and data related to said moisture content of said wood material is transferred wirelessly to a remote device.

Some preferable embodiments of the invention are described in the dependent claims.

In one embodiment, the impedance data is alternatively or additionally provided between the first depth and the second depth by measuring impedance between at least one electrode of the first pair and the second pair. This embodiment may enable to recognize, if one or more electrodes of the measuring means are in contact with a defect part and/or this embodiment may enable a correction of measurement data based on measured impedance data between electrodes in different depths. The embodiment in question may be able to detect one or more defect parts near surface, for example, and/or deeper in the wood material in question and/or in a different layer of the wood material.

In an embodiment, the cross-measurement between electrodes at different depths is performed prior to actual measurements for detecting defect parts and/or assuring the performance of the measuring means, i.e. for performing a self-check.

In one embodiment wherein a distance between electrodes in said first pair and/or said second pair is selected to be at least 20 mm, preferably at least 30 mm, and most preferably at least 40 mm, and in another embodiment, a distance between said first pair and said second pair is selected to be at least 20 mm, preferably at least 30 mm, and most preferably at least 40 mm.

In another embodiment, the distances between electrodes in a pair and/or between the pairs are selected based on quality specifications of the wood material in question, for example. Typically, the quality specifications define the hardness of the wood material in question and/or the size of defect parts, such as knots, for example. In addition, the allowed places for knots may be defined.

Naturally, the person skilled in the art will understand that the maximum distance between the electrodes is limited by the size of the measured wood material.

These embodiments may help to deduct if an electrode is in contact with a defect part by possibly preventing more than one electrode from coming into contact with the same defect part in the wood material. In addition, these embodiments may help prevent measurement along and/or across a grain of the wood material. Typically, other defect parts than knots in wood material occur along a grain, so preferably electrodes are avoided to locate in the same grain.

In an embodiment, pairs of electrodes are placed with each other in a diamond formation when viewed from above. This embodiment may enable to prevent the electrodes from being positioned along and/or across a grain of the wood material in any depths and/or layers.

It may be possible to avoid the situation, where more than one electrode would be placed in a same defect part, by using the diamond formation, since defect parts typically occur parallel with grains of the wood. The diamond formation may also ensure that the electrodes are not placed across a grain of the wood material in any depths and/or layers, which can improve the reliability of the measurements.

In an embodiment the diamond formation is combined with a cross-measurement between the electrodes in different depths. By detecting a difference between two cross-measurements, this embodiment may also enable to detect defects, which may be invisible being in an inner layer, as could be in Cross-Laminated Timber (CLT) or Laminated Veneer Lumber (LVL)-wood material, for example. In an embodiment, the measuring means further comprises one or more common electrodes, which are arranged to be able in an electrical connection in several depths and/or layers.

In one embodiment, said impedance investigation is a resistance measurement.

In one embodiment, at least one of electrodes may be used for fixing said body part on a surface of said wood material.

In an embodiment, the measuring means further comprises means for measuring other quantities, such as air humidity and/or temperature, for example. Especially, information relating to temperatures under zero may affect to moisture content measurement, and thus the temperature is preferably detected.

The embodiment in question comprising a temperature measurement can be advantageous, because temperature affects the resistivity of the wood and thus it can have a significant effect on the moisture content measurement results. It may be possible to compensate the effect of temperature by using correction coefficient table, which itself is known in the prior art.

Especially, a long-term measurement of relative humidity combined with temperature may provide valuable information on the condition of the wooden material in question. Relative humidity information combined with temperature information may reveal if the moisture in the wooden material is due to circumstances or weather variations. It may also provide information on the direction of moisture inflow, i.e. if the material has been in contact with water on the same side with the moisture measuring device or the moisture has come from the opposite side, for example.

In one embodiment, an air gap is arranged between said wood material and said body part. This embodiment may help to remove humidity between the body part and the surface of the wood material, and thus it may help to provide data that are more accurate. In addition, the embodiment may help to diminish the measurement to be disturbed by the body part itself.

In one embodiment, said measuring means further comprises transferring means for, advantageously wirelessly, transferring data related of the moisture content of said wood material. In an embodiment, the transferring means comprise a transmitter connectable to wireless IoT data network, such as Lora network or NB-IoT network.

Significant advantages can be achieved with the present invention when compared to the prior known solutions. With the moisture measuring device according to the present invention it may possible to measure the moisture content from several depths in a wood material. The moisture content measurement may also be possible to perform between different depths.

An electrode of the present invention may also be able to accurately arrange to a desired depth in the wood material to be measured by using electrodes with desired length, for example.

The moisture measuring device according to the present invention is especially useful for a long-term measurement and it may also be able to provide information on the moisture content of the material over a longer period of time, such as storage, transportation and construction, for example.

In addition, the moisture measuring device according to the present invention may also to provide moisture content information from a wood material comprising more than one layer by using electrode pairs in different depths. Electrode pair of a surface layer(s) may be able to detect rapid changes in the moisture content, and the deeper electrodes may be able to provide information on temporal average moisture values.

With the term “measuring means” in this application is referred to electrodes and electronics functionally connected to each other making it possible to measure and/or transfer data to a remote device.

With the term “wood material” in this application is referred to a various kinds of lumber, timber, and wood products, such as cross-laminated timber (CLT) and laminated veneer lumber (LVL), for example, and other wood material typically used as a building material, including also engineered wood material, such as plywood, for example, as well as layered structures and materials comprising wood, such as composite materials, for example.

Term “defect part” in this application refers to various kinds of impurities in wood, such as, but not limited to knot parts, black knot parts, knot holes, pith parts and resin pockets, for example.

### SHORT DESCRIPTION OF THE DRAWINGS

Next, the invention is described in more detail with reference to the appended drawings, in which

FIG. 1 illustrates a top view of a body part and measuring means according to an embodiment of the present invention,

FIG. 2 illustrates a side view of a body part and measuring means according to an embodiment of the present invention,

FIG. 3 illustrates a side view of an electrode according to an embodiment of the present invention,

FIG. 4 illustrates a block diagram of a system according to an embodiment of the present invention, and

FIG. 5 illustrates a flow diagram of an exemplary method of the invention.

### DETAILED DESCRIPTION OF THE EMBODIMENTS

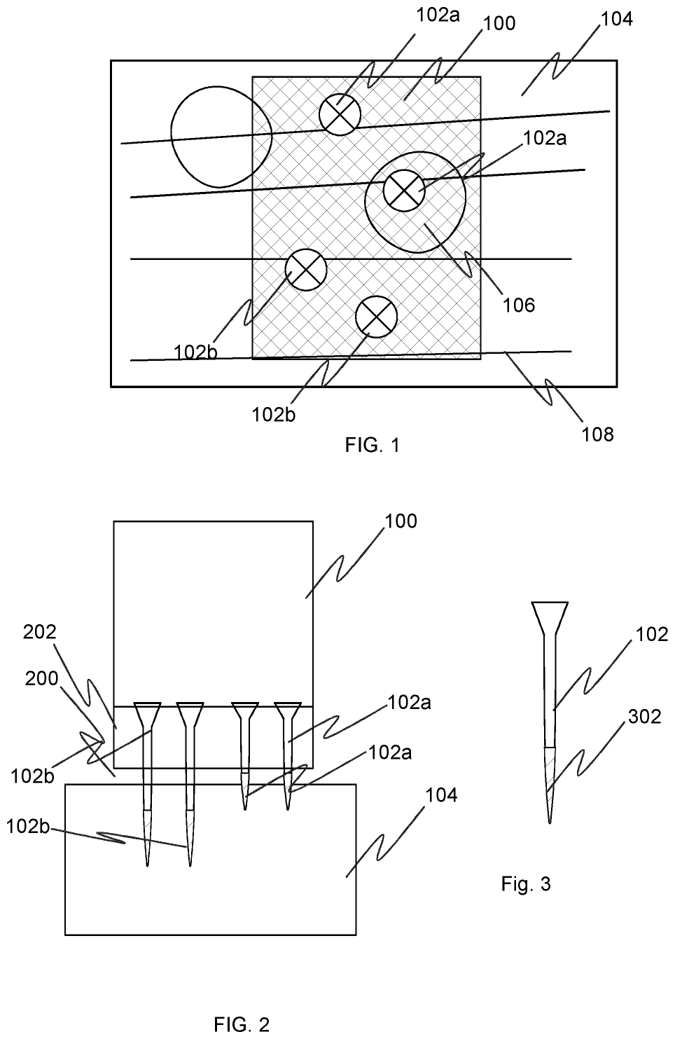

FIGS. 1 and 2 illustrate a top view and a side view, respectively, of a body part 100 and measuring means according to an embodiment of the present invention. In an embodiment, the body part 100 of the present invention comprises measuring means, i.e. required measuring electronics for executing investigating impedance of a wood material to be measured. The solutions for measuring impedance are known as such and are not explained more carefully in this document.

In an embodiment, a part of the measuring means are arranged separately from the body part, such as a jig part, for example, which is functionally connected to the measuring means in the body part. The person skilled in the art will understand that the jig part can comprise suitable part of the measuring means, such as, but not limited, electrodes separately or in a pair, and wires connected the electrodes to the measuring means, for example.

The size and shape of the body part 100 of the present invention can be of any size and shape suitable for the purpose. The optimal size and shape of the body part depends on a desired distance between electrodes 102a, 102b of the measuring means. The thickness depends on size of measuring means including required electronics and battery.

The material used in the body part 100 may be any material suitable for the purpose, e.g. plastic such as ABS (Acrylonitrile butadiene styrene), metal such as aluminum, rubber, silicone, glass and/or porcelain. It is obvious to a person skilled in the art that these materials may be used separately or they may be combined in a favorable way.

The body part 100 is preferably arranged on a surface of a wood material 104 to be measured. In an embodiment, measuring means, such as one or more electrodes 102 are used for fixing the body part 100 to the wood material 104. In an embodiment, electrodes 102 have a shape of screws for fixing the body part to the wood material, as can be seen in FIG. 2.

In an embodiment, the body part further comprises guiding means 202 for guiding electrodes 102 and/or other fixing means into a right location and/or an angle. The person skilled in the art will understand that the guiding means can be performed in various ways, such as arranging a form of a cone in the guiding means for guiding a pin shape electrode to a right location.

In an embodiment, an air gap 200 is arranged between the wood material 104 and body part 100. The width of the air gap 200 can vary, but preferably it is at least 1 mm, for example, for help to remove humidity between the body part 100 and the surface of the wood material 104.

The measuring means according to the present invention comprises at least a first pair of electrodes 102a and a second pair of electrodes 102b to be inserted in the wood material 104 for investigating impedance of the wood material 104. The person skilled in the art will understand that the measuring means of the present invention may comprise more electrodes, single ones or in pairs than is described in this document.

In an embodiment, the impedance investigation is a resistance measurement. The resistance measurement is performed between electrodes in a pair. By inserting electrodes 102a, 102b into different depths, the measuring means may be able to provide measurement data from different layers in the wood material in question.

In another embodiment, the impedance data is alternatively or additionally provided between a first depth and a second depth by measuring impedance between at least one electrode of the first pair and the second pair. This embodiment may enable the measurement data between different depths and/or layers in the wood material in question.

Measuring the impedance between different depths by varying the measuring electrodes in a pair may also help to detect a defect part in the wood material. In an embodiment, the system of the present invention is further arrange to compensate the defect part in question in order to provide data that are more correct.

In an embodiment, the cross-measurement between electrodes at different depths is performed prior to actual measurements for detecting defect parts and/or assuring the performance of the measuring means, i.e. for performing a self-check.

Further, a measuring depth of the first pair 102a of electrodes and said second pair of electrodes 102 b is arranged to differ for providing impedance data from a first depth and/or a second depth of the wood material 104.

In an embodiment, a distance between the electrodes in the first pair 102a and/or the second pair 102b is selected to be at least 20 mm, preferably at least 30 mm, and most preferably at least 40 mm. The distance is selected to better avoid locating more than one electrode in the same defect part 106 in the wood material 104.

In an embodiment, the electrodes in the first pair 102a and/or the second pair 102b are positioned aside from parallel and/or perpendicular line relative to each other in order to better avoid measurement along and/or across a grain 108 of the wood material 104.

In an embodiment, a distance between said first pair 102a and said second pair 102b is selected to be at least 20 mm, preferably at least 30 mm, and most preferably at least 40 mm.

In an embodiment, the first pair 102a and the second pair 102b are positioned aside from parallel and/or perpendicular line relative to each other in order to better avoid measurement along and/or across a grain 108 of the wood material 104, when measuring between different depths.

In an embodiment, the first pair 102a of electrodes and the second pair 102b of electrodes are placed with each other in a diamond formation when viewed from above, as can be seen in FIG. 1.

In an embodiment the diamond formation is combined with a cross-measurement. The diamond formation may ensure that the electrodes are not positioned along and/or across a grain of the wood material in any depths and/or layers. It could be possible to detect a defect part in a deeper layer, for example, by using cross measurement of electrodes in different depths in the diamond formation and comparing the results of the two cross-measurements. Significant variation in the results suggests a defect part, which may be in an inner layer, for example. In an embodiment, the measuring means are further arranged to provide one or more moisture content values based on measured impedance data.

In an embodiment, the measuring means further comprises means for measuring other quantities, such as air humidity and/or temperature, for example.

FIG. 3 a side view of an electrode 102 according to an embodiment of the present invention. The person skilled in the art will understand that the size and the shape of electrodes in the present invention may vary, but preferably electrodes are pin shaped or screw shaped.

In an embodiment, the electrode 102 comprises fixing means, such as threads, for example, for fixing the body part into a surface of the wood material and/or for inserting the electrode into a desired measuring depth in the wood material.

In an embodiment, the length of the electrodes in the first pair differs from the length of the electrodes in the second pair. In an embodiment, wherein the electrodes are used for fixing the device in question on a surface of the wood material to be measured, the electrodes are arranged in desired measuring depths and/or layers when fixing the device.

In an embodiment, the electrode 102 comprises an insulation layer on its outer surface arranged in such a way, that a part 302 of the electrode is left uninsulated for providing measurement data from a desired depth, when inserted into the wood material to be measured. The location of the uninsulated part 302 may vary. In the embodiment, depicted in FIG. 3, the uninsulated part 302 is arranged at the tip of the electrode 102.

In the embodiment in question, the length of the electrodes in different pairs typically vary for providing measurement data from different depths and/or layers of the wood material.

In another embodiment, the uninsulated part 302 is arranged at some other part of the electrode 102, such as to be located near the surface of the wood material, when inserted. This embodiment can enable usage of the same length of electrodes in different pairs, but still providing measurement data from different depth of the wood material.

In an embodiment, the measuring means further comprises one or more common electrodes, which are arranged to be able in an electrical connection in several depths and/or layers. The electrode according to the embodiment in question can be arranged totally without insulation or the insulation is arranged in such a way that the electrode is capable for electrical connection in various depths and/or layers.

In an embodiment, the desired depth of one or more electrodes is achieved by using supporting means, such as one or more washers, baseplates, support chairs, bedplates, spacers, for example, but not limited. In this way, it may be possible to arrange the electrode in question in a desired depth and/or layer in a wood material, but adjusting the length of the shaft of the electrode.

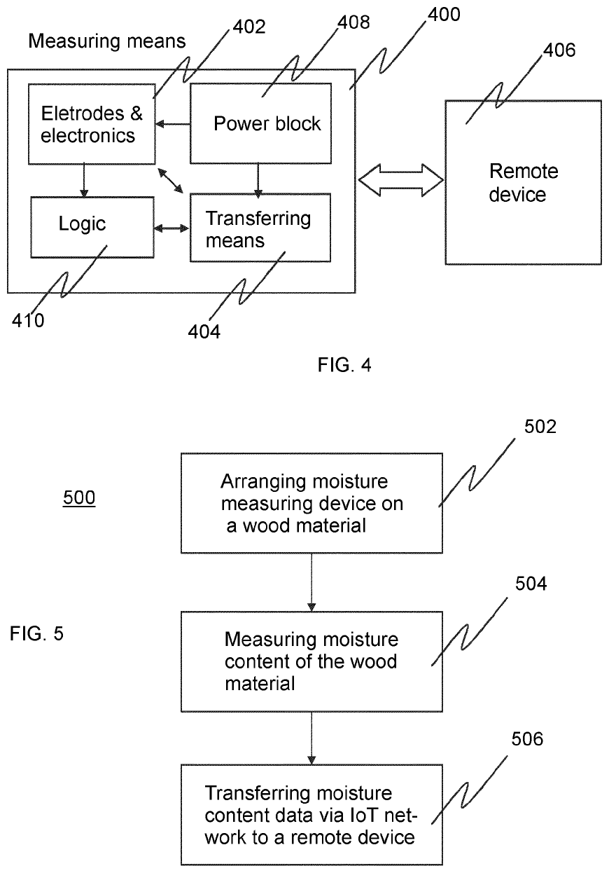

FIG. 4 illustrates a block diagram of a system according to an embodiment of the present invention. measuring means 400 according to the present invention comprises required electrodes and electronics 402 functionally connected to each other making it possible to measure the moisture content of a wood material in question in desired depths.

According to an embodiment, the measuring means 400 further comprises transferring means 404 functionally connected in the measuring means for transferring measured impedance data and/or one or more moisture content values to a remote device 406. The transferring means of the present invention may be implemented in various ways. In an embodiment, the transferring means comprises a transmitter connectable to wireless IoT network, such as Lora network or NB IoT network.

In one embodiment the transferring means is provided on a conventional circuit board. The transferring means may also be covered with epoxy. In one embodiment of the invention, the transferring means are provided directly in the epoxy, without a circuit board. In another embodiment, the transferring means may be split on multiple circuit boards. In a further embodiment, certain parts of the transferring means may be on the circuit board and some parts may be separate without circuit board.

In an embodiment, the measuring means further comprises a logic block comprising typical components, such as, but not limited to, a processor, one or more arithmetic blocks and/or memory, for example. Preferably, logic means are used for providing one or more moisture content values based on measured impedance data.

In an embodiment, the system of the present invention further comprises a remote device 406 arranged to at least receive measurement data from the moisture measuring device according to the present invention. The person skilled in the art understand that the type of the remote device may vary being a computer, a cloud service, a mobile device, a smart phone and/or a handheld device, for example depending on technique of transferring means.

In an embodiment, wherein the data is transferred to a proximity reader, the device in question is advantageously placed in an easily accessible place of the structure of a wooden building. The device may not need to be visible, but it may be possible to place behind a thin plate, like a gypsum board.

In an embodiment, the transferring means is also arranged for receiving commands from a remote device in case of requirement of measurement data, for example.

In an embodiment, the measuring means further comprises energy means 408, such as battery/batteries, for providing energy to the measuring means, transferring means and/or logic means.

In an embodiment, the inductive energy feed is used for providing energy the device. This embodiment may be advantageous in cases where the device is meant to be used for occasionally moisture content checks, for example.

FIG. 5 illustrates a flow diagram of an exemplary method 500 according to the present invention. First at step 502, the moisture measurement device according to the present invention is arranged on a wood material to be measured. At step 504, the measuring means of the measurement device are arranged to measure the moisture content of wood material. At step 506, the measured moisture content data is transferred to a remote device.

The present invention can also be used for measuring a moisture content of layered structures and materials comprising wood, such as composite materials, for example. The device according to the present invention can be arranged in layered structures and/or materials comprising wood, e.g. a wooden frame plasterboard wall, where the device can be mounted on a finished panel wall surface to measure the frame wood in a lower layer, for example.

It should also be noted that although the device is embedded in a horizontal structure in the above embodiments, it is naturally possible to use the invention also for structures in other orientations, such as vertical structures. In such case the device has a corresponding orientation relative to the surface of the structure. The scope of the invention is determined by the attached claims together with the equivalents thereof. The skilled persons will again appreciate the fact that the explicitly disclosed embodiments were constructed for illustrative purposes only, and the scope will cover further embodiments, embodiment combinations and equivalents that better suit each particular use case of the invention.

1. Device for measuring moisture content of a wood material, comprising:
a body part arrangeable on a surface of said wood material,
measuring means being functionally connected to the body part, said measuring means comprising at least a first pair of electrodes and a second pair of electrodes to be inserted in said wood material for investigating impedance of said wood material,
wherein a measuring depth of said first pair of electrodes and said second pair of electrodes is arranged to differ for providing impedance data from a first depth and/or a second depth of said wood material.

2. The moisture measuring device according to claim 1, wherein said impedance data is alternatively or additionally provided between said first depth and said second depth by measuring impedance between at least one electrode of said first pair and said second pair.

3. The moisture measuring device according to claim 2, wherein said impedance data measured between said first depth and said second depth is used for determining if an electrode is arranged in a defect part.

4. The moisture measuring device according to claim 1, wherein a distance between electrodes in said first pair and/or said second pair is selected to be at least 20 mm.

5. The moisture measuring device according to claim 1, wherein a distance between said first pair and said second pair is selected to be at least 20 mm.

6. The moisture measuring device according to claim 1, wherein pairs of electrodes are placed with each other in a diamond formation.

7. The moisture measuring device according to claim 1, wherein said impedance investigation is a resistance measurement.

8. The moisture measuring device according to claim 1, wherein at least one of said electrodes is used for fixing said body part on a surface of said wood material.

9. The moisture measuring device according to claim 1, wherein an air gap is arranged between said wood material and said body part.

10. The moisture measuring device according to claim 1, wherein said device further comprises transferring means for transferring data related of the moisture content of said wood material.

11. Measuring system for measuring moisture content of a wood material, wherein the system comprises a measuring device according to claim 1, means for receiving and collecting data related to a moisture content of said wood material via IoT network.

12. Method for measuring moisture content of a wood material, wherein a moisture measuring device according to claim 1 is arranged on a wood material to be measured, moisture content of said wood material is measured with said moisture measuring device, and data related to said moisture content of said wood material is transferred via wireless IoT data network for analysis of the data.

13. The moisture measuring device according to claim 1, wherein a distance between electrodes in said first pair and/or said second pair is selected to be at least 30 mm.

14. The moisture measuring device according to claim 1, wherein a distance between electrodes in said first pair and/or said second pair is selected to be at least 40 mm.

15. The moisture measuring device according to claim 1, wherein a distance between said first pair and said second pair is selected to be at least 30 mm.

16. The moisture measuring device according to claim 1, wherein a distance between said first pair and said second pair is selected to be at least 40 mm.

17. The moisture measuring device according to claim 2, wherein said impedance investigation is a resistance measurement.

18. The moisture measuring device according to claim 3, wherein said impedance investigation is a resistance measurement.

19. The moisture measuring device according to claim 4, wherein said impedance investigation is a resistance measurement.

20. The moisture measuring device according to claim 5, wherein said impedance investigation is a resistance measurement.
